(19) United States
(12) Patent Application Publication
(10) Pub. No.: US 2011/0270051 A1
(43) Pub. Date: Nov. 3, 2011

(54) Cardiovascular Disease Screening Method and Apparatus

(76) Inventors: Morteza Naghavi, Houston, TX (US); Albert Andrew Yen, Pearland, TX (US); Haider Ali Hassan, Houston, TX (US); David S. Panthagani, Houston, TX (US)

(21) Appl. No.: 13/099,606

(22) Filed: May 3, 2011

Related U.S. Application Data
(60) Provisional application No. 61/343,680, filed on May 3, 2010.

(51) Int. Cl. A61B 5/00

(52) U.S. Cl. 600/301

(57) ABSTRACT

The disclosure teaches non-invasive, inexpensive and reproducible tests that provide improved measurement of risk assessment by measurement of the following parameters of a subject. The disclosure includes intima-media thickness, augmentation index, arterial wall elasticity, central arterial pressure, electrocardiogram impedance, cardiograph blood pressure measurement, ankle-brachial index, 3D (three dimensional) plaque volume, vessel wall volume, and diameter waveform pattern characterization. Further, the disclosure teaches that satisfactory measurement of risk assessment may be achieved by conducting any four of the aforementioned tests.

### RELATED APPLICATION

This Application claims the benefit of and claims priority to Provisional Application Ser. No. 61/343,680 entitled Cardiovascular Disease Screening Method and Apparatus filed May 3, 2010. Application 61/343,680 is incorporated herein by reference in its entirety.

### FIELD OF THE INVENTION

The present invention relates generally to the field of assessing a patient's cardiovascular health. More particularly, the invention relates to a method of providing a comprehensive cardiovascular assessment of a patient by associating functional, risk factor, and structural assessments of the patient's cardiovascular system. The disclosure also includes an apparatus for performing the testing and measurements to allow the assessment.

### BACKGROUND OF THE INVENTION

Cardiovascular disease (CVD) is the leading cause of death in the United States and most developed countries. The epidemic of CVD is growing fast in the developing countries as well as the under privileged part of developed societies who cannot afford advanced and often expensive diagnostic and therapeutic modalities. It is now well documented that almost all cases of CVD are due to atherosclerotic cardiovascular disease and manifest predominantly by heart attack and stroke. The unpredictable nature of heart attack and the need for cost-effective screening in large groups of asymptomatic at-risk populations are unsolved problems in cardiovascular healthcare.

Risk Factor Based Risk Assessment:

In the past 50 years, although numerous risk factors for atherosclerosis have been identified, the ability to predict a cardiovascular event, particularly in the near term, remains elusive. Numerous population studies have shown that over 90% of CVD patients have one or more risk factors (high cholesterol, blood pressure, smoking, diabetes etc.). However, 70-80% of the non-CVD population also has one or more risk factors. Over 200 risk factors have been reported, including a number of emerging serologic markers. For example, lipid profiles (Total cholesterol, LDL, HDL, triglycerides), homocysteine, and C-reactive protein (CRP) have been adapted for coronary risk assessment.

High blood cholesterol is a major risk factor for coronary heart disease and stroke. Cholesterol plays a major role in a person's heart health. The National Cholesterol Education Program (NCEP) has guidelines for detection and treatment of high cholesterol. The Third Report of the Expert Panel on Detection, Evaluation, and Treatment of High Blood Cholesterol in Adults (Adult Treatment Panel III or ATP III) was released in 2001. It recommends that everyone age 20 and older have a fasting “lipoprotein profile” every five years. This blood test is performed after a 9-12-hour fast without food, liquids or pills. It gives information about total cholesterol, LDL cholesterol, HDL cholesterol and triglycerides. Based on combining this lipoprotein information with a Framingham Risk Score (FRS), the NCEP has developed thresholds to guide initiation of therapeutic lifestyle changes and/or drug therapy.

The FRS is a coronary prediction algorithm that seeks to provide an estimate of total coronary heart disease (CHD) risk (risk of developing one of the following: angina pectoris, myocardial infarction, or coronary disease death) over the next 10 years. Separate score sheets are used for men and women, and the factors used to estimate risk include age, total blood cholesterol, HDL cholesterol, blood pressure, cigarette smoking, and diabetes mellitus. Relative risk for CHD is estimated by comparison to low-risk Framingham participants of the same age, optimal blood pressure, total cholesterol 160-199 mg/dL, HDL cholesterol 45 mg/dL for men or 55 mg/dL for women, non-smoker and no diabetes. The Framingham Heart Study risk algorithm encompasses only coronary heart disease (CHD), not other heart and vascular diseases, and was based on a study population that was almost all caucasian. Wilson P W F, et al. “Prediction of coronary heart disease using risk factor categories” Circulation 97 (1998) 1837-1847. In addition, the Framingham Risk Score is heavily weighted by age and sex and thus has low predictive value for individuals under 55 and for women.

A sensitive screening test for early atherosclerotic vascular disease should correlate with the magnitude of Framingham Risk Estimates, and should predict CHD vs. absence of CHD. However, Framingham risk estimates are intended to predict risk of future CHD events, not presence of CHD. A >20% 10-year estimated risk is regarded as “CHD-equivalent.” It is noteworthy that new guidelines consider diabetes as a “CHD equivalent.” An incremental predictive value over FRS for CHD suggests a complementary or alternative clinical utility and provides an impetus for the present invention.

Further, a recent guideline has brought to light the need for direct and individualized assessment of cardiovascular health, beyond the mere assessment of risk factors. (Naghavi et al. From Vulnerable Plaque to Vulnerable Patient. Executive Summary of the Screening for Heart Attack Prevention and Education (SHAPE) Task Force Report. The American J. of Cardiology. Supplement to vol 98, no. 2. July 17, 2006). As highlighted in the SHAPE Guideline, current primary prevention recommendations from initial assessments and risk stratification are based on traditional risk factors (e.g., the Framingham Risk Score in the United States and the SCORE in Europe), followed by goal-directed therapy when necessary. Although this approach may identify persons at very low or very high risk of a heart attack or stroke within the next 10 years, the majority of the population belongs to an intermediate-risk group, in which the predictive power of risk factors is low. Indeed, most heart attacks occur in this intermediate-risk group.

Consequently, many individuals at-risk will not be properly identified and will not be treated to attain appropriate “individualized” goals. Others will be erroneously classified as high risk and may be unnecessarily treated with drug therapy for the rest of their lives. (See also Akosah K, et al., “Preventing myocardial infarction in the young adult in the first place: how do the National Cholesterol Education Panel III guidelines perform?,” J Am Coll Cardiol. 2003 May 7;41(9):1475-9; Brindle P, et al. “Predictive accuracy of the Framingham coronary risk score in British men: prospective cohort study,” BMJ, 2003 Nov 29, 327 (7426):1267; Empana J P, et al., “Are the Framingham and PROCAM coronary heart disease risk functions applicable to different European populations? The PRIME Study,” Eur Heart J. 2003 Nov 24(21):1903-11; Neuhauser H K, et al. “A comparison of Framingham and SCORE-based cardiovascular risk estimates in participants of the German National Health Interview and Examination Survey 1998,” Eur J Cardiovasc Prey Rehabil, 2005 Oct 12(5):442-50; Bastuji-Garin S, et al., “Intervention as a Goal in Hypertension Treatment Study Group. The Framingham prediction rule is not valid in a European population of treated hypertensive patients,” J Hypertens. 2002 Oct 20(10):1973-80.) In short, the predictive accuracy of risk factor analysis, when performed alone in a given individual, is poor. The SHAPE Guideline highlights the need for structural and functional assessment of the arterial system, in addition to risk factor analysis, and also recognizes insufficiencies in available tools for structural and functional assessments of atherosclerosis.

Functional Status of the Cardiovascular System:

Assessment of cardiovascular function has focused on the endothelial system. Endothelial function (EF) is accepted as a sensitive indicator of vascular function. EF has been labeled a “barometer of cardiovascular risk” and is well-recognized as the target of cardiovascular disease. Endothelial cells comprise the innermost lining of the vasculature. In addition to forming a physical barrier, endothelial cells play a central role in multiple regulatory systems including vasomotion, inflammation, thrombosis, tissue growth and angiogenesis. When there is increased demand for blood by organs of the body, endothelial cells release nitric oxide (NO), which increases the diameter of arteries and thereby increases blood flow. Nitric oxide is important not only for the regulation of vascular tone but also for its roles in the modulation of cardiac contractility, response to vessel injury, and development of atherosclerosis. Presence of atherosclerosis hampers the normal functioning of these cells, blocking NO-mediated vasodilation and making the arteries stiffer and less able to expand and contract. The loss of ability of an artery to respond to increased and sudden demand is called endothelial dysfunction (EDF).

Endothelial dysfunction is associated with virtually all of the cardiovascular risk factors, and endothelial failure is the end stage that leads to clinical events in cardiovascular disease. Numerous experimental, clinical, and epidemiologic studies have shown that endothelial function is altered in the presence of established risk factors such as hypertension, hypercholesterolemia, diabetes mellitus and emerging risk factors such as hyperhomocysteinemia, CRP, and fibrinogen. Evidence showing strong correlations between endothelial dysfunction and other sub-clinical markers of atherosclerosis, such as carotid intima media thickness (IMT), coronary calcium score (CCS), and ankle brachial index (ABI), has also emerged. More importantly, endothelial dysfunction has been reported to be predictive of coronary, cerebro-vascular and peripheral arterial disease and can be detected before the development of angiographically significant plaque formation in the coronary and peripheral vasculature by measuring the response to pharmacological and physiological stressors. Endothelial function not only predicts risk, it also tracks changes in response to therapy (pharmacologic and non-pharmacologic) and alterations in risk factors.

Traditional techniques for assessment of endothelial function are invasive, and include: forearm plethysmography with intra-arterial acetylcholine challenge testing; cold pressor tests by invasive quantitative coronary angiography; and injection of radioactive materials and mapping blood flow by tracing movement of radiation. The invasive nature of these tests limits widespread use, particularly in the asymptomatic population. Non-invasive methods include: measurement of the percent change in diameter of the left main trunk induced by cold pressor test with two-dimensional (2-D) echocardiography; the Dundee step test measuring the blood pressure response of a person to exercise (N Tzemos, et al. Q J Med 95 (2002) 423-429); laser Doppler perfusion imaging and iontophoresis; high resolution B-mode ultrasound to study vascular dimensions (T J Anderson, et al. J. Am. Col. Cardiol. 26(5) (1995) 1235-41); occlusive arm cuff plethysmography (S Bystrom, et al. Scand J Clin Lab Invest 58(7) (1998) 569-76); and digital plethysmography or peripheral arterial tonometry (PAT)(A Chenzbraun et al. Cardiology 95(3) (2001) 126-30). Of these, brachial artery imaging with high-resolution ultrasound (BAUS) during reactive hyperemia is considered the gold standard method of assessing peripheral vascular function. Brief, suprasystolic arm cuff inflation provides an ischemic stimulus. Ischemia reduces vascular resistance in the tissues distal to cuff occlusion, and cuff release is accompanied by a sudden rise in blood flow (reactive hyperemia). The increased blood flow through the brachial artery elicits dilation of the arterial wall. Ultrasound imaging of the diameter of the artery, along with measuring the peak flow, defines endothelial function. However, this BAUS method requires very sophisticated equipment and operators that are only available in a few specialized laboratories worldwide. Thus, despite widespread use of BAUS in clinical research, technical challenges, poor reproducibility, and considerable operator dependency have limited the use of this technique to vascular research laboratories.

Venous occlusion plethysmography evaluates peripheral vasomotor function by measuring volume changes in the forearm by mercury strain gauges during hyperemia. A recent review of plethysmography suggested that this method is poorly reproducible, highly operator-dependent, time consuming, and cumbersome. (Yvonne-Tee, G B, et al. “Noninvasive assessment of cutaneous vascular function in vivo using capillaroscopy, plethysmography and laser-Doppler instruments: its strengths and weaknesses,” Clin Hemorheol Microcirc. 2006;34(4):457-73. Review.) Tissue doppler imaging or flowmetry of the hand can be employed to continuously show skin perfusion before and after hyperemia using single fiber/point Doppler measurement of flow at finger tip. These techniques are also expensive and limited in availability. Alternatively, peripheral arterial tonometry (PAT) can be used to measure changes in the volume of finger as the indicator of changes in blood flow which in turn reflects changes in the diameter of brachial artery during hyperemia. This method is non-invasive but is not inexpensive and is not conducive to self-administration.

Structural Status of the Cardiovascular System:

Structural tests that are available include an array of diagnostic tests that directly evaluate the presence or physical effects of atherosclerosis and/or CVD. Such structural tests include carotid intimal-medial thickness (IMT) and plaque measurements by ultrasound, aortic and carotid plaque detection by magnetic resonance imaging (MRI), coronary calcium scoring by CT, and peripheral vascular disease detection by ankle-brachial index (ABI) measurement. These tests are valuable for detection of existing conditions and disease progression but are expensive, difficult to self-administer, not easily repeatable, and lack predictive value of vascular reactivity and early stage atherosclerosis.

“Vascular Age”:

A few studies have suggested that some of these structural tests can be used to determine an individual's “vascular” and/or “coronary” age, to use in place of the individual's chronological age, and thereby improve cardiovascular risk estimation. (Stein J H et al. “Vascular age: Integrating carotid intima-media thickness measurements with global coronary risk assessment,” Clinical Cardiology 2004; 27:388-392; Enrique F Schisterman et al., “Coronary age as a risk factor in the modified Framingham risk score,” BMC Med Imaging. 2004; 4: 1. Published online 2004 April 26. doi: 10.1186/1471-2342-4-1.) However, even newer data has shown that high coronary calcium scores and/or carotid IMT measures are indicative of existing atherosclerotic cardiovascular disease, so the substitution of a ‘vascular age’ or ‘coronary age’ variable in risk prediction models may not be necessary. Also, structural tests are more beneficial for identification and treatment of existing disease than for primary prevention, as they are only capable of visualizing existing disease when there are already high levels of coronary calcium, IMT, and/or atherosclerosis. An effort of the present invention is to provide a direct and comprehensive assessment of vascular age (both function and structure) during all stages of atherosclerosis to enhance the identification, prevention, and/or treatment of CVD.

Accordingly, existing cardiovascular risk assessments face limitations in detection, treatment, devices, and administration. What is needed is a non-invasive, inexpensive and reproducible apparatus that provides improvement in measurement of risk assessment by combining risk factor, functional, and structural assessments of cardiovascular health.

### DETAILED DESCRIPTION OF DISCLOSURE

The instant disclosure teaches non-invasive, inexpensive and reproducible tests that provide improved measurements of risk assessment. The test measures the following listed characteristic of major arteries, including but not limited to the Carotid and Femoral arteries.

The disclosure includes intima-media thickness (measurement of wall thickness of the arteries which can detect the presence and tracks progression of atherosclerotic disease and correlates to cardiovascular disease), and augmentation index. The disclosure also includes arterial wall elasticity, central arterial pressure, electrocardiogram impedance, cardiograph blood pressure measurement, ankle-brachial index, 3D (three dimensional) plaque volume, vessel wall volume, and diameter waveform pattern characterization. Further, the disclosure teaches that satisfactory measurement of risk assessment may be achieved by conducting any four of the aforementioned tests.

The invention utilizes automated Carotid IMT measurement. Measured is the intima-media thickness of the artery wall thickness to detect the presence and tracks progression of atherosclerotic disease and correlates the measured results to cardiovascular disease.

Also utilized is automated plaque detection, i.e., the build up of cholesterol, fat, calcium, and other blood components in arteries and the presence of plaque that can lead to coronary heart disease.

Further clarification (high priority, AIDA1) utilizes a cross sectional view to avoid lateral wall blind spots. Situations may exist where plaque is visible but AIDA is not capable of measuring it. Scanning can be performed by circumferential sweep and address calcified plaque/spot (shadowing).

The disclosure also utilizes shear stress imaging performed by Doppler flow velocity, ultrasound imaging of arterial anatomy, and computational fluid dynamics (CFD). Hemodynamics and vessel geometry have been linked to pathogenesis of atherosclerosis. Areas of low and/or oscillating wall shear stress appear to be more vulnerable. A shear stress map could locate vulnerable anatomies where rapid plaque development is more likely to occur.

The disclosure includes the use of 3D modeling of velocity to generate a flow model. A flow model can be used to calculate shear stresses and formulate shear stress map of an artery.

The disclosure also includes using ultrasound imaging/3D mapping. This allows elasticity analysis, based on measurement of arterial strain using ultrasound signals, to assess risk. Arterial elasticity is the ability of an artery to expand and contract with cardiac activity. Reduced arterial elasticity is a risk factor for atherosclerosis coronary heart disease.

The disclosure utilizes an augmentation index to measure central arterial stiffness as an indicator of cardiovascular disease. It can be correlated with IMT and ΔP/PP*100 relating augmentation index and Pulse Wave Velocity in the assessment of arterial stiffness. Augmented systolic pressure represents wave reflections caused by arterial thickness.

The disclosure additionally teaches basing the augmentation index on the ratio of peak systolic diameter and peak diastolic diameter of the vessel, extracted from the vessel diameter waveform. The disclosure further teaches the central arterial pressure is measured in conjunction with brachial blood pressure and a Valsalva or Mueller maneuver.

Brachial Artery FMD evaluates endothelial dysfunction which may be correlated to early subclinical stage atherosclerotic disease and cardiovascular risk. The evaluation measures a percentage difference of the basal diameter and hyperemic diameter of the brachial artery. The procedure measures the diameter of the brachial artery by ultrasound, occludes the brachial artery with a sphygmomanometer (i.e., systolic plus 50 mm Hg) causing ischemia and followed by deflation of the sphygmomanometer and measured diameter during hyperemia.

The brachial artery diameter can be measured using an ultrasound probe. Diameter measurements can be made once before occlusion and once after occlusion. Alternatively, the diameter can be measured before occlusion then the diameter monitored during or after the procedure to find maximum diameter. This procedure may evaluate the additional parameters of calculated rate to reach maximum diameter and rate to return to normal diameter.

The disclosure further teaches that 3D volumetric assessment is performed in conjunction with measurement and calculation of total plaque burden, longitudinal area, 3D plaque volume measurement, and vessel wall volume.

Three dimensional graphics of the volumetric assessment (3D volumetric assessment) may be rendered with 3D graphics for visual representation of the various parameters. The disclosure teaches use of 3D volumetric assessment to monitor changes and progression of disease.

In one embodiment, the disclosure teaches measurement and calculation of brachial artery diameter prior to conducting cuff induced hyperemia protocol as well as after conducting the protocol and during the conduct of a cuff induced hyperemia protocol using an automated vessel diameter calculation algorithm such as the one provided by the Panasonic CardioHealth® Station (CardioNexus Corporation, Houston, Tex.). An example of this embodiment would be a device similar in appearance to the Panasonic EW3153W Diagnostec™ Arm-in Cuffless Blood Pressure Monitor with an embedded ultrasonic probe (such as the one included in the Panasonic CardioHealth® Station) capable of continuously measuring the brachial artery diameter.

In another embodiment taught by the disclosure, the diameter waveform pattern characterization, measured by analysis of ultrasound signals, depends upon the analysis of the waveform morphology and amplitude. Other arterial parameters calculated from the diameter waveform and Doppler flow velocity signals, include but are not limited to: resonance of the artery, vascular impedance based on velocity and diameter, impedance spectrum, reflection coefficient, and forward and backward waveforms.

Femoral and Popliteal artery IMT/plaque screening can be performed with an option to perform fIMT with some fine tuning of an auto-freeze algorithm criteria required, i.e., waveform, ROI height. An example of an auto-freeze algorithm/function is provided by the Panasonic CardioHealth® Station (CardioNexus Corporation, Houston, Tex.).

Abdominal Aortic IMT measurement involves a decrease in transmission frequency to achieve measurement using the same CHS probe (9.3 mHz center). The disclosure utilizes an auto-freeze algorithm with fine tuning.

Impedance Cardiography (ICG) and Pulse Wave Velocity involves using ICG for measuring cardiac output and ventricular function to monitor patients with heart failure. The disclosure may also utilize pulse wave velocity for vascular stiffness.

The disclosure includes cardiohealth station comprising arm blood pressure test capabilities, blood tests (lipid panel, Hs-CRP, proteomic marker for near event heart attacks, EKG (12 lead) for detection of arrhythmias, and ankle brachial index test to diagnose peripheral arterial disease.

This specification is to be construed as illustrative only and is for the purpose of teaching those skilled in the art the manner of carrying out the invention. It is to be understood that the forms of the invention herein shown and described are to be taken as the presently preferred embodiments. As already stated, various changes may be made in the shape, size and arrangement of components or adjustments made in the steps of the method without departing from the scope of this invention. For example, equivalent elements may be substituted for those illustrated and described herein and certain features of the invention maybe utilized independently of the use of other features, all as would be apparent to one skilled in the art after having the benefit of this description of the invention.

While specific embodiments have been illustrated and described, numerous modifications are possible without departing from the spirit of the invention, and the scope of protection is only limited by the scope of the accompanying claims.

1. A method for assessment of cardiovascular health, comprising the measuring and calculating of at least four of the following parameters in a subject:
a) Intima-Media thickness;
b) Augmentation Index;
c) Arterial wall elasticity;
d) Central arterial pressure;
e) Electrocardiogram;
f) Blood pressure measurement;
g) Ankle-brachial Index;
h) 3D (three dimensional) vessel wall and plaque volume; and
i) Diameter waveform pattern characterization.

2. The method in claim 1 wherein the parameters include:
a) Intima-Media thickness;
b) Arterial wall elasticity;
c) 3D (three dimensional) vessel wall and plaque volume; and
e) Diameter waveform pattern characterization.

3. The method in claim 1 where the Augmentation Index is based on the ratio of peak systolic diameter and peak diastolic diameter of the vessel, extracted from the vessel diameter waveform.

4. The method in claim 1 where the central arterial pressure is measured in conjunction with brachial blood pressure and a surrogate measure of carotid pressure waveform is measured by carotid ultrasound.

5. The method in claim 1 where the 3D volumetric assessment is performed in conjunction with measurement and calculation of:
a) Total plaque burden;
b) Longitudinal surface area of plaque;
c) 3D plaque volume measurement; and
d) Vessel wall volume.

6. The method in claim 4 where 3D volumetric assessment is rendered with 3D graphics for visual representation of the various parameters.

7. A method of using 3D ultrasonic volumetric assessment of arterial wall to monitor response to therapy and changes (progression or regression) in status of cardiovascular disease.

8. An apparatus and automated method of utilizing ultrasonic signals to continuously measure and calculate brachial artery diameter pre, post, and during a cuff induced reactive hyperemia protocol for assessment of vascular function as an indicator of cardiovascular health.

9. The method in claim 1 where the diameter waveform pattern characterization depends upon the analysis of the waveform morphology and amplitude as well as additional arterial parameters calculated from the diameter waveform and Doppler flow velocity signals, including but not limited to:
a) Resonance of the artery;
b) Vascular impedance based on velocity and diameter;
c) Impedance spectrum;
d) Reflection coefficient; and
e) Forward and backward waveforms.

10. An apparatus for assessment of cardiovascular health, comprising components for the measurements and calculations of the following parameters in a subject:
a) Intima-Media thickness;
b) Augmentation Index;
c) Arterial wall elasticity;
d) Central Arterial pressure;
e) Electrocardiogram;
f) Blood pressure measurement;
g) Ankle-brachial Index;
h) 3D (three dimensional) vessel wall and plaque volume; and
i) Diameter waveform pattern characterization.

11. The apparatus of claim 10 further comprising an impedance cardiograph.

12. The method and apparatus for measuring a diameter of a brachial artery by evaluating the measured percentage difference of a basal diameter and hyperemic diameter of the brachial artery by measuring the diameter of the brachial artery by ultrasound, occluding the brachial artery with a sphygmomanometer, causing ischemia and followed by deflation of the sphygmomanometer and measuring the brachial artery diameter during hyperemia.
